(19) United States
(12) Patent Application Publication
(10) Pub. No.: US 2012/0009265 A1
(43) Pub. Date: Jan. 12, 2012

(54) DRUG CARRIER

(76) Inventors: Chih-Hui Yang, Chiayi City (TW); Keng-Shiang Huang, Chiayi County (TW)

(21) Appl. No.: 12/834,499

(22) Filed: Jul. 12, 2010

(51) Int. Cl. A61K 9/14; A61P 35/00

(52) U.S. Cl. 424/489; 977/773; 977/906; 977/810

(57) ABSTRACT

A drug carrier comprises a substance, a paramagnetic particle, a rod-shaped light-absorbing particle and a drug. The paramagnetic particle, the drug and the rod-shaped light-absorbing particle absorbing light and generating heat are provided in the substance.

### BACKGROUND OF THE INVENTION

1. Field of the Invention

The present invention relates to a drug carrier, particularly to a drug carrier with multiple utilities of magnetic targeting, photothermal therapeutic, drug releasing and tumor recognition.

2. Description of the Related Art

In general speaking, a conventional drug carrier comprises a particular polymer in order to perform a coat of a therapeutic drug, wherein the particular polymer can be a thermosensitive polymer, a pH-sensitive polymer or a photosensitive polymer for specifically releasing the therapeutic drug in biological creatures.

A conventional drug carrier as defined in Taiwan paten no. I314465 discloses a thermosensitive nanostructure for hyperthermia treatment, which comprises a thermosensitive polymer nanostructure presented a coat of a paramagnetic nanoparticle of ferric oxide and a drug, with a lower critical solution temperature (LCST) of around 40 to 45. In this way, the paramagnetic nanoparticle of ferric oxide can produce thermoenergy to heat the thermosensitive polymer nanostructure till 40 to 45 under a driving of an additional magnetic field for processing of thermomagnetic treatment. Meanwhile, when the temperature of the thermosensitive polymer nanostructure goes up higher than the LCST, the structural collapse of the thermosensitive polymer nanostructure and the drug release may happen for approaching to thermomagnetic therapeutic and medicinal treatment.

Another conventional drug carrier as defined in Taiwan patent no. 200900082 discloses a drug carrier comprises a thermosensitive liposome with lipid bilayer encapsulating a paramagnetic nanoparticle of ferric oxide and a drug. In this way, the delivery of the encapsulated paramagnetic nanoparticle of ferric oxide and drug can be carried out by the thermosensitive liposome to a proposed affected part, followed by heating up with a magnetic field in order to thermal-degraded the structure of the thermosensitive liposome for the drug releasing within a thermomagnetic effect.

However, the conventional drug carriers as disclosed in Taiwan patent no. I314465 and 200900082 need an additional strong magnetic field to initiate the thermomagnetic effect on the paramagnetic nanoparticle of ferric oxide which may only be available in hospital. As a result, for most users, it is less convenient for self-operating the thermomagnetic effect in private houses but necessarily require a professional equipment of strong magnetic field. Furthermore, due to the strong magnetic field demanded for initiating the thermomagnetic effect, motions of some electronic drives, such as a heart pacemaker, installed in users' bodies will be interfered, which may be harmful to users' life.

The other drug carrier as define in Taiwan patent no. 200623170 discloses a drug carrier in a core-shell structure comprises a magnetic particle as a magnetic core with a coat of a seeding layer. Wherein the seeding layer, a shell component with character of optical absorption is formed via a reduction between a metal and the seeding layer, which can be a gold shell, a platinum shell or a sliver shell. In this way, the core-shell structure is capable to apply in biomedical diagnosis, such as magnetic resonance imaging (MRI) developer, for magnetic conducting the core-shell structure to a proposed affected part. Furthermore, in accord with a thermomagnetic efficiency involved in an additional magnetic field, the drug carrier provides a thermomagnetic treatment to the proposed affected part. On the other hand, the shell component with an optical absorption band can specifically absorb lights in particular wavelength to apply to thermal treatment of cancer cells.

However, the conventional drug carrier as disclosed in Taiwan patent no. 200623170 needs a seeding layer placed on the surface of the magnetic core for producing the shall component via the reduction. Hence, the manufacturing process of the conventional drug carrier is complicated. Next, when the diameter of the magnetic core is larger than 40 nm, the shell component may easily escape from the magnetic core. Also, the shell component is performed at uniform depth which can only absorb a single wavelength of light to produce thermal energy in a less efficient manner.

Though what mentioned above, there is a necessity of improving the disadvantages of the conventional drug carrier.

### SUMMARY OF THE INVENTION

The primary objective of this invention is to provide a drug carrier, which has multiple utilities of magnetic targeting, photothermal therapeutic, drug releasing and tumor recognition.

The secondary objective of this invention is to provide a drug carrier, which can avoid interference from additional strong magnetic field with motions of some electronic drives installed in users' bodies.

Another objective of this invention is to provide a drug carrier which can transact and absorb two wavelengths of light to perform photothermal therapeutic efficiency.

Another objective of this invention is to provide a drug carrier which is convenient for self-operation.

Another objective of this invention is to provide a drug carrier, which can produce thermal energy in an efficient manner.

A drug carrier comprises a substance, a paramagnetic particle, a rod-shaped light-absorbing particle and a drug. The paramagnetic particle, the drug and the rod-shaped light-absorbing particle absorbing light and generating heat are provided in the substance.

Further scope of the applicability of the present invention will become apparent from the detailed description given hereinafter. However, it should be understood that the detailed description and specific examples, while indicating preferable embodiments of the invention, are given by way of illustration only, since various will become apparent to those skilled in the art from this detailed description.

### BRIEF DESCRIPTION OF THE DRAWINGS

The present invention will become more fully understood from the detailed description given hereinbelow and the accompanying drawings which are given by way of illustration only, and thus are not limitative of the present invention, and wherein:

FIG. 1 is a diagram illustrating a structure of a drug carrier in the present invention;

FIG. 2 is an IVIS imaging system of a rat before a treatment of the drug carrier in the present invention;

FIG. 3 is an IVIS imaging system of a rat after a treatment of the drug carrier in the present invention; and

FIG. 4 is an IVIS imaging system of a rat after a treatment of the drug carrier and illumination of a near infrared light in the present invention.

In the various figures of the drawings, the same numerals designate the same or similar parts.

### DETAILED DESCRIPTION OF THE INVENTION

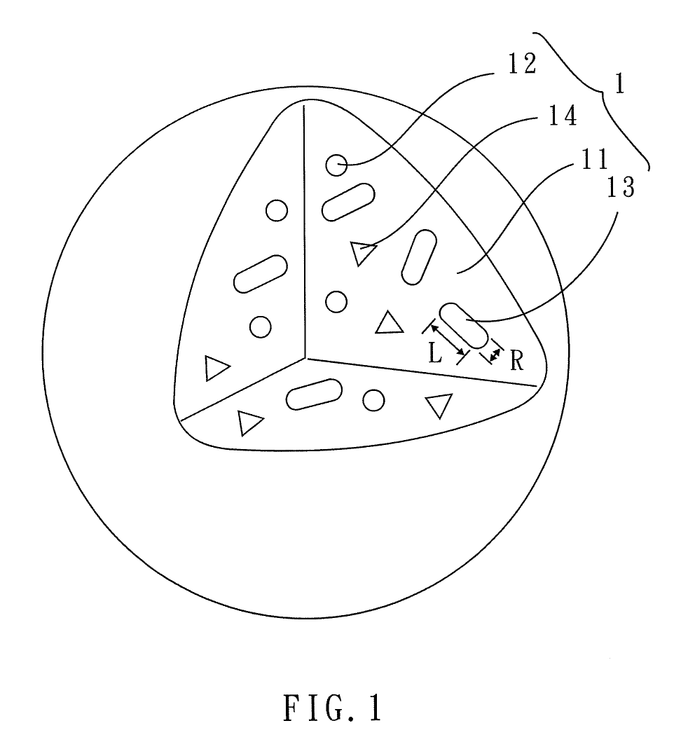

Referring to the FIG. 1, in accordance with a preferable embodiment of a drug carrier 1 in the present invention comprises a substance 11, at least one paramagnetic particle 12, a rod-shaped particle 13 and a drug 14.

According to the FIG. 1, the substance 11 made from any polymer with particular function, such as thermosensitive polymers, pH-sensitive polymers and photosensitive polymers, which can be selected from a group of PEG, PLGA, PLA, PGA, PCL and PMMA. As an example, the substance 11 in the preferable embodiment of the drug carrier 1 in the present invention is made from a complex of PCL and PLGA which contains a higher glass transition temperature (T_{g}) than the temperature at a proposed area for drug delivering in order to avoid an unexpected structural variation of the substance 11 before achievement of delivery. In additional, the diameter of the substance 11 is preferring distributed over 50 to 500 nm to exclude an interference with the transposition of the drug carrier 1 caused by accumulating precipitation of the drug carrier 1.

As shown in the FIG. 1, the paramagnetic particle 12 is embodied inside the substance 11, wherein, the paramagnetic particle 12 can be any super paramagnetic materials like ferric, cobalt, nickel, ferric oxide, ferric cobalt, ferric nickel or other paramagnetic materials. In this way, the magnetism of the paramagnetic particle 12 will only be observed under the control of additional magnetic field. As an example, the paramagnetic particle 12 in the preferable embodiment of the drug carrier 1 in the present invention is made from a paramagnetic ferric oxide (Fe₃O₄) with a preferring diameter from 5 to 50 nm can significantly avoid an accumulating precipitation happened. In accord with the magnetism of paramagnetic particle 12 installed inside the substance 11, the drug carrier 1 in the present invention can be magnetic conducted and condensed in bodies, also precisely transported to the proposed areas to perform in an magnetic targeting manner. For example, the paramagnetic ferric oxide (Fe₃O₄) used in the preferable embodiment of the drug carrier 1 in the present invention can be a MRI contrast mecium for detecting the distribution of the drug carrier 1 in bodies, therefore, a real-time photoanalytic system of drug release can be established. Hence, with the drug carrier 1 in the present invention, not only can achieve the magnetic targeting effects, but also can further apply the magnetic targeting performance to tumor recognition for the sake of directly treating of tumor cells in the proposed area.

Referring to the FIG. 1, the rod-shaped particle 13 is embodied inside the substance 11 which has optical absorption of ultraviolet (UV), lights in near infrared region (NIR), far infrared region and visible lights (VIS) and further transfer absorbed lights into thermal energy. The rod-shaped particle 13 can be made from gold, platinum and sliver with a preferring ratio between the length (L) and diameter (R) of the rod-shaped particle 13 diverse from 2 to 5. Additionally, due to the diversity between the length (L) and the diameter (R) of the rod-shaped particle 13, it is allow of absorbing two wavelengths of lights at the same time, therefore, the drug carrier 1 can produce thermal energy in a more efficient way. For example, the rod-shaped particle 13 in the preferable embodiment of the drug carrier 1 in the present invention is made from gold, with 37.2±5.4 nm in length (L), 9.4±5.4 nm in diameter (R) and 3.9 in the ration of (L)/(R) which can absorb NIR lights both in wavelength of 480 nm to 550 nm and 760 nm to 820 nm.

As shown in the FIG. 1, the drug 14 also embodied inside the substance 11 can be various according to a proposed disease for interest. As an example, the drug 14 in the preferred embodiment of the drug carrier 1 in the present invention is selected from anti-tumor drugs, such as cisplatin, but not limited to that. In this way, the drug 14 (cisplatin for example) can precisely move to the proposed area via the magnetic conduction of the paramagnetic particle 12 of the substance 11 followed by illumination of NIR lights in order to induce the release of the drug 14 for treating of tumor cells in the proposed area as recognized.

In summary, the substance 11 contains the paramagnetic particle 12, the rod-shaped particle 13 and drug 14. In the present invention, the drug carrier 1 can be guided to a proposed area via a magnetic conduction of the paramagnetic particle 12 under magnetic attractions of an additional magnetic field to perform in a magnetic targeting manner. Moreover, with the illumination of NIR lights on the proposed area, the rod-shaped particle 13 of the drug carrier 1 can absorb photoenergy of lights distributed in two wavelengths and further transfer the photoenergy into thermoenergy for approaching to photothermal therapy. Meanwhile, the thermoenergy can heat up the substance 11 till higher than the glass transition temperature (T_{g}) which may result in the structural collapse of the substance 11 and release of the drug 14 to achieve a facility in selective drug releasing.

Through the present invention, the collapse of the substance 11 and the release of the drug 14 are preformed under photothermal but thermomagnetic efficiency, therefore, an strong additional magnetic filed for conducting the drug releasing will no longer be needed. In this situation, the users may only require an electromagnet to initiate the motion of the drug carrier 1 in any time and any place. Hence the drug carrier 1 in the present invention can not only avoid the safety issue caused by strong magnetic field, also bring about more convenience to the users.

On the other hand, the drug carrier 1 in the preferable embodiment of a drug carrier 1 in the present invention is manufactured under the following processes. First of all, an organic solution and a water solution are prepared and mixed up with the drug 14. Generally, it is preferred for a water-soluble drug to dissolve in the water solution, a fat-soluble drug to dissolve in the organic solution. For example, a fat-soluble anti-tumor drug, tamoxifen, PCL/PLGA and ferric oxide (Fe₃O₄) in a ratio of 2:27:4.95 are prepared and dissolved in chloroform wherein the PCL/PLGA and ferric oxide (Fe₃O₄) are differentially used in the manufacture of the substance 11 and the paramagnetic particle 12. In the present invention, the ratio between the drug, PCL/PLGA and ferric oxide (Fe₃O₄) can be further adjusted according to the selection of the drug. Additionally, a water-soluble anti-tumor drug, epirubicin, and gold nanoparticles in a ratio of 1:1 are dissolved in deionized water, wherein the gold nanoparticles are used as the rod-shaped particles 13 with 37.2±5.4 nm in length (L), 9.4±1.8 nm in diameter (R) and a ratio of L/R in 3.9.

Next, one ration of the organic solution and ten rations of the water solution are mixed and emulsified with each other under a processing of blender to obtain a mixture. As following, 10 ml of 1% polyvinyl alcohol (PVA) are sequentially dropped into 1 ml of the mixture while shaking of a sonicator for 15 minutes for secondary emulsification, wherein the PVA is performed in a surfactant to assist with the process of the secondary emulsification. While the organic solution has evaporated completely via continuously stirring, a suspension can be collected.

Finally, the suspension is repeatedly processed in a cycle of washing by water, centrifugation and taking pellet in order to completely remove residue organic solutions. Accordingly, the drug carrier 1 in the present invention will be obtained which contains multiple utilizations of magnetic targeting, photothermal therapeutic, drug releasing and tumor recognition as shown above. In the present invention, the drug carrier 1 is preferred to resuspend in 1 ml of deionized water for further application.

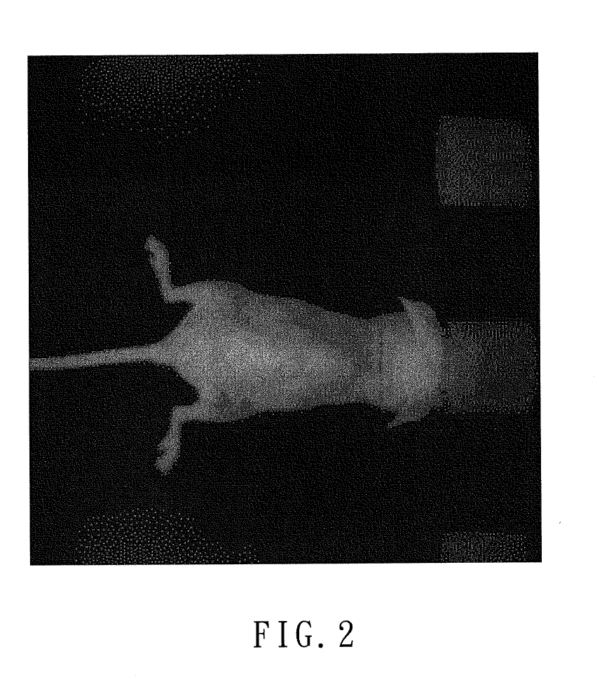

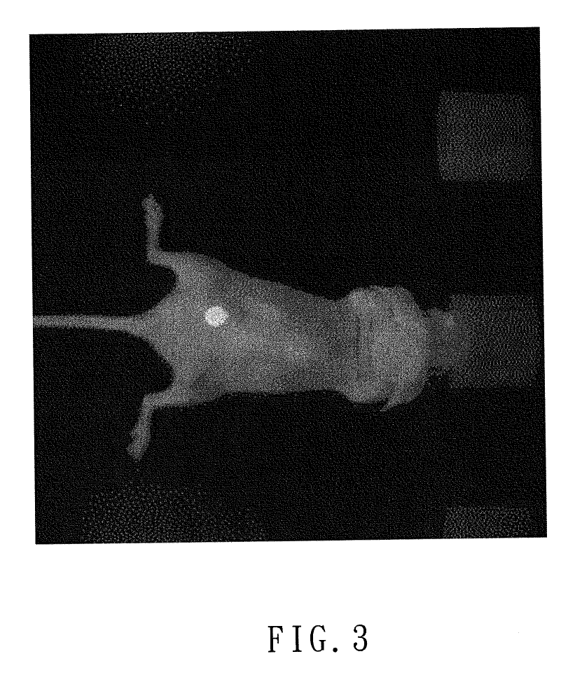

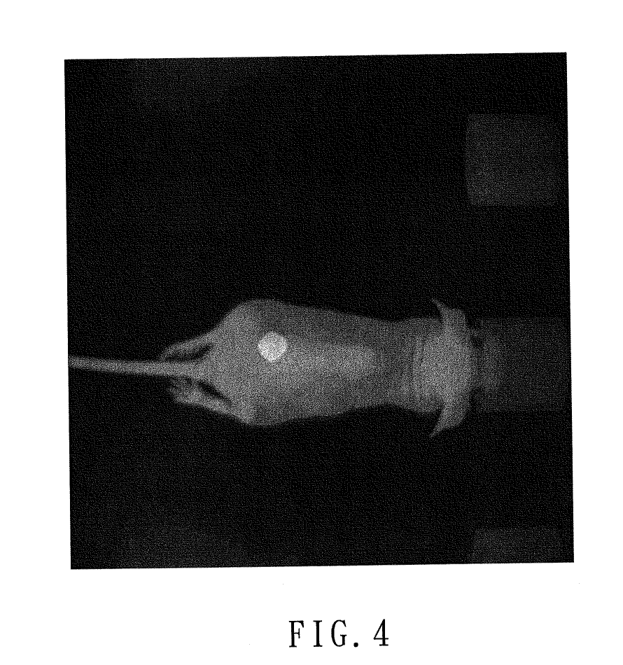

Referring to FIGS. 2, 3 and 4 separately, shows an IVIS imaging systems of an animal model before (FIG. 2), or after (FIGS. 3 and 4) treating of the drug carrier 1 in the present invention. As an example, 100 μl of the drug carrier 1 are subcutaneous injected into a rat, also 5000 Hz of magnetic field provided from an electromagnet are supplied to the rat for magnetic conducting the motion of drug carrier 1. As shown in the FIG. 3, the drug carrier 1 is aggregating at a target region via the magnetic attraction of the magnetic field for the paramagnetic particle 12 with a magnetic targeting manner. Furthermore, according to the FIG. 4, while the drug carrier 1 finally collected at the target region, the drug carrier 1 can be heated up through illumination of NRI lights to perform in a photothermal therapeutic manner. The NRI lights used in the present invention is short in wavelength but strong in penetration, which is adequate to penetrate through the skin of rat and directly act on the drug carrier 1. According, the temperature of the drug carrier 1 will go up by absorbing two wavelengths of lights to achieve the photothermal therapeutic efficiency.

Also, a higher temperature (higher than the T_{g} of the substance 11, PCL/PLGA) will lead to the structural collapse of the substance 11, therefore, the drug 14 will be released carrying out the drug releasing and the tumor-recognition efficiency, while illustrating in the FIG. 4. Therefore, it is suggested that the drug carrier 1 in the present invention show multiple abilities of magnetic targeting, photothermal therapeutic, drug releasing and tumor-recognition.

Through the present invention, a drug carrier is provide to perform magnetic targeting, photothermal therapeutic and drug releasing efficiencies via a installation of paramagnetic particle, rod-shaped particle and drug. Furthermore, the rod-shaped particle of the drug carrier is allowed to absorb two wavelengths of lights for transferring from the photo energy to thermal energy in a more efficient way. Also, according the thermal energy produced by the rod-shaped particle, a structural collapse of the drug carrier may be stimulated to achieve the drug releasing. In this way, a safety issue cause by conventional drug carriers and additional strong magnetic field will be positively avoided.

Although the invention has been described in detail with reference to its presently preferred embodiment, it will be understood by one of ordinary skill in the art that various modifications can be made without departing from the spirit and the scope of the invention, as set forth in the appended claims.

What is claimed is:

1. A drug carrier, comprising:
a substance;
a paramagnetic particle embodied in the substance;
a rod-shaped particle with light-absorbed ability, which is embodied in the substance for converting the absorbed lights into thermal energy; and
a drug embodied in the substance.

2. The drug carrier as defined in claim 1, wherein a ratio between the length and diameter of the rod-shaped particle is from 2 to 5.

3. The drug carrier as defined in claim 1, wherein the material of the rod-shaped particle is selected from a group of gold, platinum and sliver.

4. The drug carrier as defined in claim 3, wherein the rod-shaped particle is a gold nanoparticle with a ratio of 3.9 between the length and the diameter.

5. The drug carrier as defined in claim 1, wherein the material of the paramagnetic particle is selected from a group of iron, cobalt, nickel, ferric oxide, cobalt oxide and nickel oxide.

6. The drug carrier as defined in claim 1, wherein the paramagnetic particle is made from a paramagnetic material.

7. The drug carrier as defined in claim 1, wherein the diameter of the paramagnetic particle is diverse from 5 to 50 nm.

8. The drug carrier as defined in claim 1, wherein the substance is made from a polymer.

9. The drug carrier as defined in claim 8, wherein the material of the substance is selected form a group of polymers of PEG, PLGA, PLA, PGA, PCL and PMMA.

10. The drug carrier as defined in claim 1, wherein the diameter of the carrier is from 50 to 500 nm.

11. The drug carrier as defined in claim 1, wherein the drug is an anti-tumor drug.
